(19) United States
(12) Patent Application Publication
(10) Pub. No.: US 2024/0164915 A1
(43) Pub. Date: May 23, 2024

(54) METHOD AND SYSTEM FOR EVALUATING EXOSKELETON CONTROL LOGIC

(71) Applicant: ELECTRONICS AND TELECOMMUNICATIONS RESEARCH INSTITUTE, Daejeon (KR)

(72) Inventors: Woojin KIM, Daejeon (KR); HyunSuk KIM, Daejeon (KR); Daewoong CHOI, Daejeon (KR); Oh Cheon KWON, Daejeon (KR); Daesub YOON, Daejeon (KR); Seung-Jun LEE, Daejeon (KR); Hyunwoo JOE, Daejeon (KR)

(73) Assignee: ELECTRONICS AND TELECOMMUNICATIONS RESEARCH INSTITUTE, Daejeon (KR)

(21) Appl. No.: 18/333,043

(22) Filed: Jun. 12, 2023

(30) Foreign Application Priority Data
Nov. 21, 2022 (KR) 10-2022-0156664

(51) Int. Cl. A61F 2/70

(52) U.S. Cl. CPC A61F 2/70; A61F 2002/701

(57) ABSTRACT

Provided are a method and apparatus for evaluating the control logic of an exoskeleton, wherein the method includes acquiring, by a human modeling module, characteristic data of a human model, acquiring, by a device modeling module, characteristic data of an exoskeleton, verifying, by a controllability determination module, a controllability that represents whether control is performable on a target body motion based on the characteristic data of the human model and the characteristic data of the exoskeleton, performing, by a simulation module, the target body motion based on a result verification of the controllability and acquiring simulation data generated during performance of the target body motion, and analyzing, by a performance evaluation module, the simulation data.

### CROSS-REFERENCE TO RELATED APPLICATION

This application claims priority to and the benefit of Korean Patent Application No. 10-2022-0156664, filed in the Korean Intellectual Property Office on Nov. 21, 2022, the entire contents of which are incorporated herein by reference.

### BACKGROUND

### 1. Field of the Invention

The present invention relates to a method and system for evaluating the performance of the control logic of an exoskeleton that assists muscle strength or helps to generate greater power.

More specifically, the present invention relates to a method and system for evaluating the performance of the control logic of an exoskeleton as to whether the control logic operates according to a user's movement to match a developer's intention when the exoskeleton is worn by a user.

### 2. Discussion of Related Art

A conventional exoskeleton is a wearable robot developed to assist a person's muscular strength or help to generate greater power. Typically, exoskeletons are used for military, industrial, rehabilitation, and training systems.

Exoskeletons are largely divided into a passive exoskeleton including a skeleton and springs without separate sensors and actuators, and an active exoskeleton automatically driven by sensors and actuators. In particular, in active exoskeletons, control logic that analyzes the feedback of a sensor and automatically controls an actuator is very important.

Recently, a closed-loop method (force control) in which a user's intention is obtained through a sensor and an actuator is driven according to the intention is a trend that is being developed. However, in order to verify such an interaction, the exoskeleton needs to be tested while being directly worn by a user, and there is a limitation that a user's qualitative feedback needs to be reflected to improve the interaction.

Since the feedback may be subjective depending on the user, and in order for the exoskeleton to be widely used, various user groups need to be tested, which requires a long time and money.

In addition, when unfinished control logic is directly tested on a human in a development stage, safety issues may also arise. Therefore, in order to solve the above problems, there is a need for a system capable of verifying the control logic of an exoskeleton in large quantities for various human bodies.

The above information disclosed in this Background section is only for enhancement of understanding of the background of the invention, and therefore it may contain information that does not form the prior art that is already known in this country to a person of ordinary skill in the art.

### SUMMARY OF THE INVENTION

The present invention is directed to providing a system for evaluating the control logic of an exoskeleton, which is capable of verifying the control logic of an exoskeleton with respect to a single or various human bodies of various characteristics and evaluating the performance of the control logic without the exoskeleton being directly worn by a human.

The technical objectives of the present invention are not limited to the above, and other objectives may become apparent to those of ordinary skill in the art based on the following descriptions.

According to an aspect of the present invention, there is provided a method of evaluating the control logic of an exoskeleton, the method including the steps of (a) acquiring, by a human modeling module, characteristic data of a human model, (b) acquiring, by a device modeling module, characteristic data of an exoskeleton, (c) verifying, by a controllability determination module, a controllability that represents whether control on a target body motion is performable based on the characteristic data of the human model and the characteristic data of the exoskeleton, (d) performing, by a simulation module, the target body motion based on a verification result of the controllability and acquiring simulation data generated during the performing of the target body motion, and (e) analyzing, by a performance evaluation module, the simulation data.

The characteristic data of the human model may include at least one of a length of each body part, a joint range of motion, a maximum muscle strength, and a minimum muscle strength.

The characteristic data of the exoskeleton may include at least one of a sensor wearing position, a type of sensor, a type of actuator, an actuator range of motion, a maximum torque of an actuator, a minimum torque of an actuator, and information about a target body motion.

The step (c) may include modeling an integrated system of the exoskeleton and the human model to derive a state space equation and determining controllability and observability in an available range of the exoskeleton and the human model through the state space equation.

The step (d) may include receiving, by the simulation module, control logic to be evaluated, performing a simulation corresponding to the target body motion based on the control logic to be evaluated, and acquiring interaction information between the human model and the exoskeleton during the performing of the simulation.

The acquiring of the interaction information between the human model and the exoskeleton may include acquiring data over time of a state variable related to an interaction that occurs during the performing of the simulation.

The step (e) may include performing at least one of a joint trajectory analysis, a motion delay analysis, a motion torque analysis, and a motion electromyography (EMG) analysis based on an interaction between the human model and the exoskeleton occurring during the performing of the simulation.

The joint trajectory analysis may be an operation of comparing an acquired joint trajectory graph with a predetermined value to evaluate whether the acquired joint trajectory graph maintains a certain deviation from a previously determined trajectory graph and whether the acquired joint trajectory graph smoothly continues.

The motion delay analysis may be an operation of evaluating whether a motion is delayed as much as a delay intended by a user.

The motion torque analysis may be an operation of evaluating whether a torque of acquired torque information is constant and whether the acquired torque information exceeds a specific limit value.

According to an aspect of the present invention, there is provided an apparatus for evaluating the control logic of an exoskeleton, the apparatus including a human modeling module configured to acquire characteristic data of a human model, a device modeling module configured to acquire characteristic data of an exoskeleton, a controllability determination module configured to determine whether control of a target body motion is performable based on the characteristic data of the human model and the characteristic data of the exoskeleton, a simulation module configured to perform the target body motion based on a result of the determination as to whether control of the target body motion is performable, and acquire simulation data generated during the performing of the target body motion, and a performance evaluation module configured to analyze the simulation data.

The controllability determination module may be configured to model an integrated system of the exoskeleton and the human model to derive a state space equation and determine controllability and observability in an available range of the exoskeleton and the human model through the state space equation.

The simulation module may be configured to receive control logic to be evaluated; perform a simulation corresponding to the target body motion based on the control logic to be evaluated and acquire interaction information between the human model and the exoskeleton during the performing of the simulation.

The simulation module may be configured to acquire data over time of a state variable related to an interaction that occurs during the performing of the simulation to acquire the interaction information between the human model and the exoskeleton.

The performance evaluation model may be configured to perform at least one of a joint trajectory analysis, a motion delay analysis, a motion torque analysis, and an EMG analysis based on an interaction between the human model and the exoskeleton occurring during the performing of the simulation.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a flowchart showing an evaluation system according to an embodiment of the present invention;

FIG. 2 is a block diagram illustrating an evaluation system according to an embodiment of the present invention;

FIG. 3 is a block diagram illustrating a configuration of a human modeling module according to an embodiment of the present invention;

FIG. 4 is a block diagram illustrating a configuration of a device modeling module according to an embodiment of the present invention; and

FIG. 5 is a block diagram illustrating a configuration of a control logic performance evaluation module according to an embodiment of the present invention.

### DETAILED DESCRIPTION OF EXEMPLARY EMBODIMENTS

In the following detailed description, the technology to be described below can have various changes and various embodiments, specific embodiments will be illustrated in the accompanying drawings and described in detail. However, this is not intended to limit the technology described below to specific embodiments, and it should be understood to include all modifications, equivalents, or substitutes included in the spirit and scope of the technology described below.

Terms such as first, second, A, B, etc. may be used to describe various elements, but the elements are not limited by the above terms, and are merely used to distinguish one element from another. For example, without departing from the scope of the technology described below, a first element may be referred to as a second element, and similarly, the second element may be referred to as the first element. Term “and/or” includes any combination of a plurality of related recited items or any of a plurality of related recited items.

In terms used in this specification, singular expressions should be understood to include plural expressions unless clearly interpreted differently in the context, and terms such as “comprising” refer to the described features, numbers, steps, operations, and components, parts or combinations thereof, but it should be understood that it does not exclude the possibility of the presence or addition of one or more other features, numbers, steps, operations, and components, parts or combinations thereof.

Prior to a detailed description of the drawings, it is to be clarified that the classification of components in the present specification is merely a classification for each component responsible for each main function. That is, two or more components to be described below may be combined into one component, or one component may be divided into two or more for each subdivided function. In addition, each component to be described below may additionally perform some or all of the functions of other components in addition to its main function, and some of the main functions of each component may be performed by other components. Of course, some of the main functions of each component may be exclusively performed by other components.

In addition, in performing a method or method of operation, each process constituting the method may occur in a different order from the specified order unless a specific order is clearly described in the context. That is, each process may occur in the same order as specified, may be performed substantially simultaneously, or may be performed in the reverse order.

Hereinafter, it is described that the apparatus for evaluating the control logic of an exoskeleton evaluates the control logic of an exoskeleton. The apparatus for evaluating the control logic of an exoskeleton is an apparatus that processes input data in a predetermined manner and performs calculations required for designing a control logic evaluation system according to a specific model or algorithm.

For example, the apparatus for evaluating the control logic of an exoskeleton may be implemented in the form of a personal computer (PC), a server on a network, a smart device, a chipset in which a design program is embedded, or the like.

In addition, the apparatus for evaluating the control logic of an exoskeleton may be implemented as an algorithm, program, or application software and installed in an apparatus for evaluating the control logic of a wearable device.

For the sake of convenience of description, the operation of the apparatus for evaluating the control logic of an exoskeleton implemented by algorithms, programs, or application software may be called a system for evaluating the control logic of an exoskeleton.

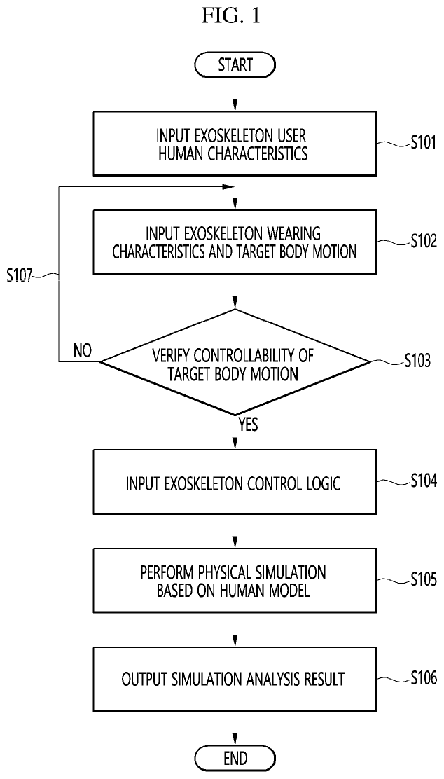

FIG. 1 is a flowchart showing an evaluation system according to an embodiment of the present invention.

Referring to FIG. 1, a system for evaluating the control logic of an exoskeleton may verify whether the control logic of an exoskeleton is applicable to various motion characteristics of humans.

First, an apparatus for evaluating the control logic of an exoskeleton according to an embodiment of the present invention may acquire the characteristics of a human model corresponding to an end-user who is to wear an exoskeleton (S101).

Specifically, the human model may be a virtual user model generated based on data acquired when a real user wears an exoskeleton.

The characteristics of the human model may include the length of each body part (an arm, leg, torso, face, etc.), a joint range of motion, a maximum muscle strength, skin characteristic information, and the like of the user who is to wear the exoskeleton.

An exoskeleton control logic evaluator may input at least one type of information among: the length of each body part, a joint range of motion, a maximum muscle strength, and skin characteristics of a user who is to wear the exoskeleton as the characteristic data of the human model, using a predetermined input value.

The apparatus for evaluating the control logic of an exoskeleton according to the embodiment of the present invention may acquire physical characteristic data of an exoskeleton and acquire information about a user's target body motion to be verified (S102).

In this case, the exoskeleton may be an exoskeleton device, and the physical characteristic data of the exoskeleton may include an exoskeleton wearing position, a type of position sensor, a type of actuator, an actuator range of motion, maximum torque information of an actuator, and the like.

The exoskeleton control logic evaluator may input at least one type of characteristics among: a wearing position, the type of sensor, the type of actuator, an actuator range of motion, and a maximum torque of an actuator into the apparatus for evaluating the control logic of an exoskeleton as physical characteristic data for evaluation. Further, the exoskeleton control logic evaluator may input information about a user's target body motion to be verified.

In this case, the target body motion may be a body motion to be evaluated by the apparatus for evaluating the control logic of an exoskeleton. For example, the target body motion may be a motion in which a specific body part and a joint move based on a real person's movement, posture, and motion, such as a “motion of bending an arm” or a “motion of bending a leg.”

The apparatus for evaluating the control logic of an exoskeleton according to the embodiment of the present invention may analyze the input characteristic data of the human model and the input physical characteristic data of the exoskeleton to perform controllability verification as to whether a range of motion, a torque limit range and the like of the human model and the exoskeleton are suitable for a target body motion (S103).

In this case, the controllability verification may be a verification in which the apparatus for evaluating the control logic of an exoskeleton determines whether an actual exoskeleton device is able to perform a target body motion based on input human characteristic data and input exoskeleton characteristic data.

For example, the controllability verification may refer to an operation of determining whether an exoskeleton device is able to perform a “motion of bending an arm” based on a muscle, a joint, a wearing position, and the like.

Specifically, the apparatus for evaluating the control logic of an exoskeleton may use sensors provided in the exoskeleton device to determine whether the exoskeleton device is correctly positioned in the position to be worn for a “motion of bending an arm” and determine whether the exoskeleton device may provide the user's muscles and joints with an appropriate force and an appropriate range of motion.

When a verification result of the controllability is that there is a problem in controllability between the input human model characteristic data and exoskeleton characteristic data and the target body motion, the apparatus for evaluating the control logic of an exoskeleton may perform a re-input request for exoskeleton physical characteristic data (S102), and acquire re-input characteristic data (S107).

For example, a case in which there is a problem in controllability between the input human model characteristic data and exoskeleton characteristic data and the target body motion may represent that parameters regarding a “motion of bending an arm” are input as the human model characteristic data and the exoskeleton characteristic data, but the target body motion corresponds to a “motion of bending a leg,” in which case the parameters of the human model characteristic data may have inputs of parameter values for an “arm” of the body, and the exoskeleton characteristic data may also have inputs of information related to a motion of an “arm” such as muscles, joints, and wearing position. In this case, since the target body motion is provided to interact with characteristic data related to a “leg,” an error may occur, and the controllability determination may indicate a result as being uncontrollable.

When the input characteristic data of the human model, the input characteristic data of the exoskeleton, and the target body motion match and no error occurs in the interaction, a controllability determination module may determine that the controllability has been verified, and request input of control logic information in which a method of driving an exoskeleton for a target motion is implemented (S104).

The evaluator may input the control logic information to be evaluated.

For example, when the target motion of the exoskeleton is a “motion of bending an arm,” the evaluator may input “arm bending motion” control logic, which is a target to be evaluated, for the “motion of bending an arm.”

An exoskeleton matched to a human model according to an embodiment of the present invention may perform a motion simulation corresponding to the target body motion based on the control logic to be evaluated.

The apparatus for evaluating the control logic of an exoskeleton may store data generated between a human modeling module and an exoskeleton device modeling module generated as the motion simulation is performed (S105).

The apparatus for evaluating the control logic of an exoskeleton according to the embodiment of the present invention may analyze the performance of the control logic based on the data that is generated between the human modeling module and the exoskeleton device modeling module and stored through the simulation, and output the analyzed data (S106).

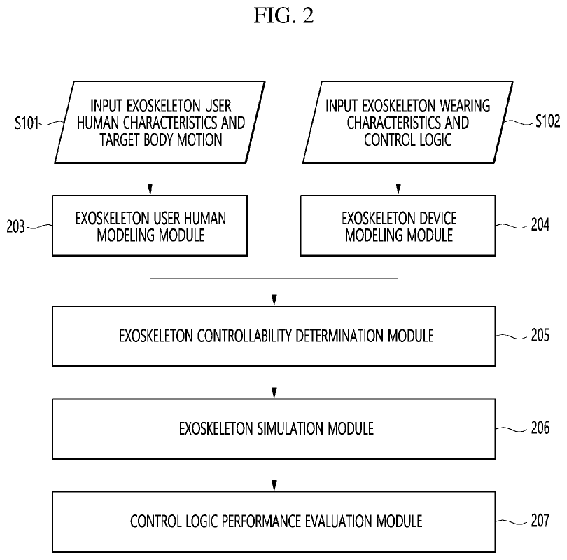

Hereinafter, components and operations of each module will be described in detail with reference to FIGS. 2 to 5.

FIG. 2 is a block diagram illustrating an evaluation system according to an embodiment of the present invention. For the sake of convenience of description, descriptions overlapping those of FIG. 1 will be omitted.

The apparatus for evaluating the control logic of an exoskeleton may include an exoskeleton user human modeling module 203, an exoskeleton device modeling module 204, an exoskeleton controllability determination module 205, an exoskeleton simulation module 206, and a control logic performance evaluation module 207.

Each of the modules may be constructed as separate hardware, such as a personal computer (PC), a server on a network, or a smart device, and may be structurally connected and implemented in the form of a chipset in which each program is embedded. In addition, the apparatus for evaluating the control logic of an exoskeleton may be implemented as an algorithm, program, or application software and installed in an apparatus for evaluating the control logic of a wearable device.

First, the evaluator may input characteristic data (a length for each body part, a joint range of motion, maximum muscle strength, etc.) of a human model corresponding to a user (an end-user) who is to wear an exoskeleton and a target body motion to be simulated into the exoskeleton user human modeling module 203.

In addition, the evaluator may input physical characteristic data (a sensor wearing position, a type of sensor, a type of actuator, an actuator range of motion, a maximum torque of an actuator, etc.) of an exoskeleton into the exoskeleton device modeling module 204 (S101), and input control logic information in which a method of driving the exoskeleton is implemented into the exoskeleton device modeling module 204 (S102).

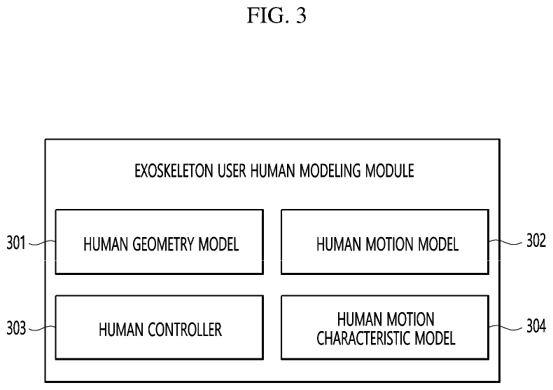

Hereinafter, the configuration and operation of the exoskeleton user human modeling module 203 will be described with reference to FIG. 3.

FIG. 3 is a block diagram illustrating a configuration of an exoskeleton user human modeling module according to an embodiment of the present invention.

Referring to FIG. 3, the exoskeleton user human modeling module 203 may include a human geometry model 301 processing information about a body length and a joint of an exoskeleton user, a human motion model 302 processing joint trajectories representing human motions, and a human controller 303 processing a method (e.g., proportional-integral-differential (PID) control, etc.) of performing motions by a human model, and a human motion characteristic model 304 processing constraints (e.g., a range of motion, maximum muscle strength, etc.) on human motions.

The exoskeleton user human modeling module 203 may perform comprehensive modeling of the exoskeleton user's body using the human controller 303 and the models 301, 302, and 304.

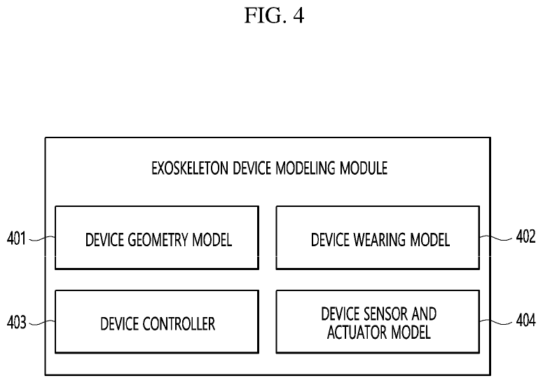

Hereinafter, the exoskeleton device modeling module 204 will be described in detail with reference to FIG. 4.

FIG. 4 is a block diagram illustrating a configuration of an exoskeleton device modeling module 204 according to an embodiment of the present invention.

Referring to FIG. 4, the exoskeleton device modeling module 204 may include a device geometry model 401 for processing information about a link and a joint of the exoskeleton device, a device wearing model 402 for processing physical information (a range of motion, wearing information, etc.) of the exoskeleton, a device controller 403 for processing the control logic of the exoskeleton, and a device sensor and actuator model 404 for processing information about a sensor and an actuator mounted in the exoskeleton.

The exoskeleton device modeling module 204 may perform comprehensive modeling on the operation of the exoskeleton device using the device controller 403 and the models 401, 402, and 404.

Hereinafter, the exoskeleton controllability determination module 205 according to the embodiment of the present invention will be described.

The exoskeleton controllability determination module 205 according to the embodiment of the present invention may be a module for determining whether control is possible in practice considering the range in which the exoskeleton device is movable, and characteristics of the sensor and the actuator based on the information from the exoskeleton user human modeling module 203 and the exoskeleton device modeling module 204.

Specifically, the exoskeleton controllability determination module 205 may perform controllability and observability analysis within a range of motion based on the characteristics of the actuator and the sensor.

In the present invention, in order to determine the controllability of the exoskeleton, the exoskeleton controllability determination module 205 may model an integrated system of the exoskeleton device and the human model to derive a state space equation, and determine controllability and observability in an available range of the exoskeleton device and the human model through the state space equation.

In this case, controllability may refer to a case in which there are consecutive inputs capable of controlling a state from an initial state to an arbitrary final state for a finite time in a control system.

Observability may refer to a case that allows a state x(t0) to be observed in a control system, given enough time during a specific time (t0≤t<tf) for information about an input u(t) and an output y(t) to determine the state x(t0).

In this case, the state space equation may refer to a mathematical model expressing a physical system as a first-order differential equation of inputs, outputs, and state variables.

That is, the terms are generally used in control systems.

Therefore, the present invention is characterized in that a state space equation is derived by modeling an integrated system of an exoskeleton device and a human model to determine the controllability of the exoskeleton.

For example, when the target motion is a “motion of bending an arm,” the exoskeleton controllability determination module 205 may determine whether the characteristic data of the human model corresponds to the “motion of bending an arm” and determine whether the characteristic data of the exoskeleton corresponds to the “motion of bending an arm.”

In addition, the exoskeleton controllability determination module 205 may determine whether the control logic input to the exoskeleton device modeling module 204 is control logic corresponding to the “motion of bending an arm.”

With such a configuration, the exoskeleton controllability determination module 205 may determine whether control of the target body motion is performable based on the characteristic data of the human model and the characteristic data of the exoskeleton.

Hereinafter, the exoskeleton simulation module 206 according to the embodiment of the present invention will be described.

The exoskeleton simulation module 206 may simulate an interaction occurring when the human model and the exoskeleton model operate according to the input control logic.

In addition, the apparatus for evaluating the control logic of an exoskeleton may store interaction data generated through the simulation as simulation data.

For example, a simulation engine may perform simulation using a physics engine, such as open dynamics engine (ODE), Bullet, Simbody, or the like.

The exoskeleton simulation module 206 may store data over time (time series data) of state variables (e.g., a force, pressure, a joint angle, a joint angle deviation, a human model electromyogram (EMG), etc. acting on each part of the human model and the exoskeleton) related to interactions occurring while performing simulation through the simulation engine, and transmit the data to the control logic performance evaluation module 207. Hereinafter, the control logic performance evaluation module 207 will be described.

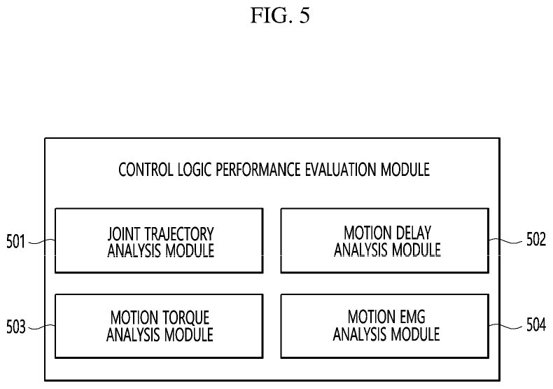

FIG. 5 is a block diagram illustrating a configuration of a control logic performance evaluation module according to an embodiment of the present invention.

Referring to FIG. 5, the exoskeleton control logic performance evaluation module 207 may include a joint trajectory analysis module 501 for analyzing information about a joint angle and a joint deviation of the exoskeleton and the human model, a motion delay analysis module 502 for analyzing the degree of delay between a joint angle of the human model and a joint angle of the exoskeleton, a motion torque analysis module 503 for analyzing a force acting between the exoskeleton and the human model, and a motion EMG analysis module 504 for analyzing the EMG of muscles generated by the human model for motion.

The control logic performance evaluation module 207 according to the embodiment of the present invention may perform performance evaluation of the exoskeleton control logic based on the interaction and the state variables transmitted through the exoskeleton simulation module 206 of FIG. 2.

For example, the motion torque analysis module 503 may evaluate torque information about whether the torque is constant and whether the torque exceeds a predetermined specific limit value. In addition, the joint trajectory analysis module 501 may evaluate whether an acquired joint trajectory graph maintains a certain deviation from a previously determined trajectory graph and whether the acquired joint trajectory graph smoothly continues by comparing the acquired joint trajectory graph with a predetermined value. In addition, the motion delay analysis module 502 may evaluate whether a delayed operation has been performed as much as a delay intended by the user.

In other words, performance evaluation may be performed by measuring and analyzing the smoothness with respect to a control input value, the efficiency, and the degree of similarity between a predetermined trajectory (a graph) and acquired data.

Those skilled in the art should appreciate that various illustrative logical blocks, modules, processors, means, circuits, and algorithm steps described in connection with the embodiments disclosed herein may be implemented as electronic hardware, various types of program or design code (for the sake of convenience, referred to as software here), or combinations thereof.

The present invention described above may be embodied as computer-readable code on a computer-readable recording medium. The computer-readable recording medium is any data storage device that can store data that can be thereafter read by a computer system. Examples of the computer-readable recording medium may include a hard disk drive (HDD), a solid-state drive (SSD), a silicon disk drive (SDD), a read-only memory (ROM), a random-access memory (RAM), a compact disc read only memory (CD-ROM), a magnetic tape, a floppy disk, an optical data storage, and the like.

As is apparent from the above, the present invention can provide a system for evaluating the control logic of an exoskeleton that is capable of verifying the control logic of an exoskeleton applicable to a single or various human bodies and evaluating the performance of the control logic, so that the control logic of an exoskeleton can be simulated without a user directly wearing the exoskeleton, and the performance of the control logic can be quantitatively evaluated based on the simulation result.

In addition, since the control logic of an exoskeleton can be developed without the exoskeleton being worn by a user, the cost and time effort incurred during development can be reduced while eliminating a concern about safety issues that can occur when verifying an exoskeleton directly worn by a user.

What is claimed is:

1. A method of evaluating control logic of an exoskeleton, the method comprising the steps of:
(a) acquiring, by a human modeling module, characteristic data of a human model;
(b) acquiring, by a device modeling module, characteristic data of an exoskeleton;
(c) verifying, by a controllability determination module, a controllability that represents whether control of a target body motion is performable based on the characteristic data of the human model and the characteristic data of the exoskeleton;
(d) performing, by a simulation module, the target body motion based on a verification result of the controllability and acquiring simulation data generated during the performing of the target body motion; and
(e) analyzing, by a performance evaluation module, the simulation data.

2. The method of claim 1, wherein the characteristic data of the human model includes at least one of a length of each body part, a joint range of motion, a maximum muscle strength, and a minimum muscle strength.

3. The method of claim 1, wherein the characteristic data of the exoskeleton includes at least one of a sensor wearing position, a type of sensor, a type of actuator, an actuator range of motion, a maximum torque of an actuator, a minimum torque of an actuator, and information about a target body motion.

4. The method of claim 1, wherein the step (c) includes:
modeling an integrated system of the exoskeleton and the human model to derive a state space equation; and
determining controllability and observability in an available range of the exoskeleton and the human model through the state space equation.

5. The method of claim 1, wherein the step (d) includes:
receiving, by the simulation module, control logic to be evaluated;
performing a simulation corresponding to the target body motion based on the control logic to be evaluated; and
acquiring interaction information between the human model and the exoskeleton as the simulation is performed.

6. The method of claim 5, wherein the acquiring of the interaction information between the human model and the exoskeleton includes acquiring data over time of a state variable related to an interaction that occurs when the simulation is performed.

7. The method of claim 1, wherein the step (e) includes performing at least one of a joint trajectory analysis, a motion delay analysis, a motion torque analysis, and a motion electromyography (EMG) analysis based on an interaction between the human model and the exoskeleton occurring when a simulation is performed by the simulation module.

8. The method of claim 7, wherein the joint trajectory analysis is an operation of comparing an acquired joint trajectory graph with a predetermined value to evaluate whether the acquired joint trajectory graph maintains a certain deviation from a previously determined trajectory graph and whether the acquired joint trajectory graph smoothly continues.

9. The method of claim 7, wherein the motion delay analysis is an operation of evaluating whether a motion is delayed as much as a delay intended by a user.

10. The method of claim 7, wherein the motion torque analysis is an operation of evaluating whether a torque of acquired torque information is constant and whether the acquired torque information exceeds a specific limit value.

11. An apparatus for evaluating control logic of an exoskeleton, the apparatus comprising:
a human modeling module configured to acquire characteristic data of a human model;
a device modeling module configured to acquire characteristic data of an exoskeleton;
a controllability determination module configured to determine whether control of a target body motion is performable based on the characteristic data of the human model and the characteristic data of the exoskeleton;
a simulation module configured to perform the target body motion based on a result of the determination as to whether control of the target body motion is performable, and acquire simulation data generated when the target body motion is performed; and
a performance evaluation module configured to analyze the simulation data.

12. The apparatus of claim 11, wherein the characteristic data of the human model includes at least one of a length of each body part, a joint range of motion, a maximum muscle strength, and a minimum muscle strength.

13. The apparatus of claim 11, wherein the characteristic data of the exoskeleton includes at least one of a sensor wearing position, a type of sensor, a type of actuator, an actuator range of motion, a maximum torque of an actuator, a minimum torque of an actuator, and information about a target body motion.

14. The apparatus of claim 11, wherein the controllability determination module is configured to:
model an integrated system of the exoskeleton and the human model to derive a state space equation; and
determine controllability and observability in an available range of the exoskeleton and the human model through the state space equation.

15. The apparatus of claim 11, wherein the simulation module is configured to:
receive control logic to be evaluated;
perform a simulation corresponding to the target body motion based on the control logic to be evaluated; and
acquire interaction information between the human model and the exoskeleton as the simulation is performed.

16. The apparatus of claim 15, wherein the simulation module acquires data over time of a state variable related to an interaction that occurs when the simulation is performed so as to acquire the interaction information between the human model and the exoskeleton.

17. The apparatus of claim 11, wherein the performance evaluation model performs at least one of a joint trajectory analysis, a motion delay analysis, a motion torque analysis, and a motion electromyography (EMG) analysis based on an interaction between the human model and the exoskeleton occurring when a simulation is performed by the simulation module.

18. The apparatus of claim 17, wherein the joint trajectory analysis is an operation of comparing an acquired joint trajectory graph with a predetermined value to evaluate whether the acquired joint trajectory graph maintains a certain deviation from a previously determined trajectory graph and whether the acquired joint trajectory graph smoothly continues.

19. The apparatus of claim 17, wherein the motion delay analysis is an operation of evaluating whether a motion is delayed as much as a delay intended by a user.

20. The apparatus of claim 17, wherein the motion torque analysis is an operation of evaluating whether a torque of acquired torque information is constant and whether the acquired torque information exceeds a specific limit value.
